(19) United States
(12) Patent Application Publication
(10) Pub. No.: US 2018/0342316 A1
(43) Pub. Date: Nov. 29, 2018

(54) DEFINITION METHOD FOR IMPLEMENTATIONS OF EXAMINATION TASKS

(71) Applicant: Siemens Healthcare GmbH, Erlangen (DE)

(72) Inventors: Lutz DOMINICK, Eggolsheim (DE); Vladyslav UKIS, Nuernberg (DE)

(73) Assignee: Siemens Healthcare GmbH, Erlangen (DE)

(21) Appl. No.: 15/981,971

(22) Filed: May 17, 2018

(30) Foreign Application Priority Data
May 23, 2017 (EP) 17172455.2

(51) Int. Cl. G16H 30/40; G16H 70/20; G16H 10/60; G16H 40/63; G16H 40/67

(52) U.S. Cl. CPC G16H 30/40; G16H 70/20; G16H 40/67; G16H 40/63; G16H 10/60

(57) ABSTRACT

A computing device accepts an examination task to be carried out by a medical imaging device from an operator. An implementation of the examination task to be realized is accepted and assigned to the examination task. Based upon the data typifying the person to be examined and data defining the type of examination of the examination task, it then establishes possible implementations of the examination task. The database contains data typifying the person, broken down according to the person to be examined, type of examination, medical imaging device and settings of the medical imaging device, a medical technology assessment of the respective examination and a commercial assessment of the respective examination. It specifies the established possible implementations together with the two for selection. Based upon the implementation to be realized, a flowchart is updated so that the flowchart contains what is now the examination task and the assigned implementation.

### PRIORITY STATEMENT

The present application hereby claims priority under 35 U.S.C. § 119 to European patent application number EP 17172455.2 filed May 23, 2017, the entire contents of which are hereby incorporated herein by reference.

### FIELD

At least one embodiment of the present invention generally relates to a definition method for an implementation of an examination task to be realized by a medical imaging device,
wherein a computing device accepts from a first operator the examination task to be carried out by the device,
wherein the examination task comprises data individualizing a person to be examined, data typifying the person to be examined and data defining the type of examination,
wherein the computing device accepts from the first operator an implementation of the examination task to be realized and assigns to the examination task the implementation of the examination task to be realized.

At least one embodiment of the present invention furthermore generally relates to a computer program, which comprises machine code that is able to be processed by a computing device, wherein the processing of the machine code by the computing device has the effect that the computing device carries out a definition method of this type.

At least one embodiment of the present invention furthermore generally relates to a computing device, wherein the computing device is programmed with a computer program of this type, so that, when in operation, it carries out a definition method of this type.

### BACKGROUND

Standardization in clinical workflow is an important trend. In particular during operation of modalities (=medical imaging systems such as for example CT scanners, MR scanners, C-arm systems) a standardized sequence of steps must often be adhered to. The larger a clinic or a hospital is, the more rigidly the steps to be adhered to are often specified. The steps are specified in order on the one hand to guarantee good results from a medical technology standpoint, such as a high image quality for example, and on the other hand to guarantee an efficient operation of the modality from a commercial standpoint.

A further aspect is in particular to avoid an examination that leads to results that cannot be evaluated, because the resulting images are of low quality. In this case a rescan, i.e. a repetition of the examination with different settings of the modality, would be required. A rescan is disadvantageous both from a medical technology standpoint—for example because of the additional radiation load or other physical and psychological stresses on the patient—and also from a commercial standpoint—more time is required for the actual desired result.

### SUMMARY

At least one embodiment of the present invention provides an efficient operation of medical imaging modalities.

At least one embodiment is directed a definition method. Advantageous embodiments of the definition method are the subject matter of the claims.

In accordance with at least one embodiment of the invention, a definition method comprises,
the computing device establishes possible implementations of the examination task on the basis of data typifying the person to be examined and the data of the examination task defining the type of examination of the examination task while accessing a database,
the database contains in each case, broken down according to data typifying the person to be examined, type of examination, medical imaging device and settings of the medical imaging device, a medical technology assessment of the respective examination and a commercial assessment of the respective examination,
the computing device specifies to the first operator for selection the established possible implementations of the examination task together with the respective medical technology assessment and the commercial assessment and
the computing device, on the basis of the implementation of the examination task to be realized accepted by the first operator, updates a flowchart for the medical imaging device, so that the flowchart, in addition to the implementations of other examination tasks already contained in the flowchart, also contains what is now the current examination task and the assigned implementation to be realized of the examination task.

At least one embodiment is further achieved by a computing device, comprising:
at least one processor configured to
   accept, via the at least one processor and from a first operator, the examination task to be carried out by the medical imaging device, the examination task including data individualizing a person to be examined, data typifying the person to be examined and data defining a type of examination method;
   accept, via the at least one processor and from a first operator, an implementation of the examination task to be realized and assigning the implementation, of the examination task to be realized, to the examination task;
   break down the computing device, according to the data typifying the person to be examined, and data defining the type of examination of the examination task, while accessing a database, to establish possible implementations of the examination task, the database containing data typifying the person, broken down according to the person to be examined, type of examination, medical imaging device and settings of the medical imaging device, a medical technology assessment of the respective examination and a commercial assessment of the respective examination;
   specify, via the at least one processor, the possible implementations of the examination task established to the first operator together with the respective medical technology assessment and the commercial assessment for selection; and
   update, via the at least one processor, based upon the implementation of the examination task to be realized accepted from the first operator, a flowchart for the medical imaging device, the flowchart, in addition to implementations of other examination tasks already contained in the flowchart, containing the examination task and the assigned implementation of the examination task to be realized.

At least one embodiment is further achieved by a non-transitory computer readable medium, storing machine code executable by a computing device, the machine code being configured to cause, when executed by the computing device, the computing device to carry out at least one embodiment of the method.

At least one embodiment is further achieved a computing device. In accordance with at least one embodiment of the invention, the computing device is programmed with at least one embodiment of an inventive computer program, so that, when in operation, it carries out at least one embodiment of an inventive definition method.

The characteristics, features and advantages described above, as well as the manner in which these are achieved, will become clearer and easier to understand in conjunction with the description of the implementation examples given below, which will be explained in greater detail in conjunction with the drawings. In the drawings, in schematic diagrams:

### BRIEF DESCRIPTION OF THE DRAWINGS

FIG. 1 shows an overview of an overall system,

FIG. 2 shows a flowchart,

FIG. 3 shows an examination task,

FIG. 4 shows a database and

FIGS. 5 to 10 show flowcharts.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

In accordance with at least one embodiment of the invention, a definition method comprises,
the computing device establishes possible implementations of the examination task on the basis of data typifying the person to be examined and the data of the examination task defining the type of examination of the examination task while accessing a database,
the database contains in each case, broken down according to data typifying the person to be examined, type of examination, medical imaging device and settings of the medical imaging device, a medical technology assessment of the respective examination and a commercial assessment of the respective examination,
the computing device specifies to the first operator for selection the established possible implementations of the examination task together with the respective medical technology assessment and the commercial assessment and
the computing device, on the basis of the implementation of the examination task to be realized accepted by the first operator, updates a flowchart for the medical imaging device, so that the flowchart, in addition to the implementations of other examination tasks already contained in the flowchart, also contains what is now the current examination task and the assigned implementation to be realized of the examination task.

This method of operation first of all guarantees that possible implementations of the examination task will be offered to the first operator of the computing device, which—if they are carried out—lead reliably to results able to be evaluated in a medically sensible manner. Furthermore this method of operation guarantees that the first operator, in the definition of the implementation of the examination task, also has in mind the required commercial aspects. Finally there is an automatic planning of the implementation of the examination task into the flowchart for the medical imaging device, wherein in particular the settings of the medical imaging device will also be incorporated into the flowchart.

Preferably, in at least one embodiment, there is provision for the computing device, on the basis of the flowchart, to establish for the medical imaging device the next implementation to be realized by this medical imaging device in each case and to specify to a second operator the settings of this medical imaging device for the next implementation to be realized in each case. On the one hand this enables the settings of the medical imaging device to be specified to the second operator, so that second operator then only has to accept the settings 1:1. Despite this the use of the human intellect by the second operator to check the medical imaging device is still maintained.

Preferably, in at least one embodiment, there is further provision for the computing device to have a data link to a control device for the medical imaging device and for the computing device to specify the settings of the medical imaging device for the next implementation to be realized in each case by the medical imaging device to the control device on the basis of a confirmation by the second operator. The 1:1 acceptance (which will always follow or at least follow as a rule) is thereby arranged very simply.

In the case of a number of medical imaging devices—in particular a number of medical imaging devices of the same type—there is preferably provision for the computing device to specify the established possible implementations of the examination task to the first operator, broken down according to the respective medical imaging device. This enables the computing device to accept a specification from the first operator as to the medical imaging device with which the examination task is to be carried out. On the one hand this can be a choice of the type of medical imaging device (for example CT scanner, MR scanner, C-arm system) and on the other hand, in the case of a number of medical imaging devices of the same type (for example a number of CT scanners) the choice of the specific medical imaging device, i.e. for example which CT scanners. Naturally a combined choice both of the type of medical imaging device and also of which of a number of medical imaging devices of the same type is also possible.

Preferably, in at least one embodiment, there is further provision for the computing device, on the basis of the flowchart for the medical imaging device, as is available to the computing device before the acceptance of the examination task, to establish for the possible implementations of the examination task a possible beginning of implementation in each case at which the respective possible implementation of the examination task can be started, and for the computing device, together with the established possible implementations of the examination task, also to show the first operator the possible beginning of implementation. This method of operation makes it possible for the first operator to estimate the waiting time that has to elapse until the result of the examination is available. This method of operation is of advantage in particular in conjunction with a number of medical imaging devices and the possibility of their selection by the first operator. If—for example—with the medical imaging device A, there is a long wait until the result of the examination is available because it is busy, whereas with the medical imaging device B the examination can begin immediately or soon, the first operator in this case can explicitly choose the medical imaging device B.

Preferably, in at least one embodiment, there is provision for the computing device, for accepting the implementation to be realized from the first operator, to accept a choice of the possible implementations. It is possible for the first operator to be able to choose the respective possible implementation just as it is, i.e. without modification. As an alternative it is possible for the first operator to still be able to make modifications before the (final) choice.

Preferably, in at least one embodiment, there is provision for the computing device to accept the actual beginning of implementation of the examination task by the medical imaging device and additionally the actual end of implementation of the examination task by the medical imaging device or the actual beginning of implementation of the next examination task by the medical imaging device. This makes it possible for the computing device to estimate from this data—i.e. the actual beginning of implementation of the examination task by the medical imaging device on the one hand and the actual end of implementation of the examination task by the medical imaging device or the actual beginning of implementation of the next examination task by the medical imaging device on the other hand—the period of time required to carry out the implementation of the examination task by the medical imaging device and on the basis of the estimation of the time, to update the commercial assessment.

In a similar way there is preferably provision, in at least one embodiment, for the computing device to evaluate the data acquired via the medical imaging device and/or to accept an assessment from a third operator. In this case it is possible for the computing device to update the medical technology assessment on the basis of the evaluation and/or the assessment accepted from the third operator. The third operator can be identical to the first operator.

For initially establishing the commercial assessment there is preferably provision, in at least one embodiment,
for the computing device to access datasets of examination tasks already carried out,
for the examination tasks already carried out each to comprise data typifying the person to be examined, the type of examination, the medical imaging device and the settings of the medical imaging devices, as well as the start times of the respective examination tasks,
for the computing device to establish, on the basis of the start times, the order of the examination tasks already carried out and to establish an estimated duration of implementation for the respective examination task and
for the computing device to take into consideration the estimated implementation durations of the examination tasks already carried out when establishing the commercial assessment.

It is possible here for the datasets evaluated within the framework of the aforementioned method of operation to have been created independently of the inventive definition method.

Preferably there is provision, in at least one embodiment,
for the database, broken down into data typifying the person to be examined, type of examination and medical imaging device, to contain a usual area in each case for the settings of the medical imaging device and/or the medical technology assessment of the respective examination and/or the commercial assessment of the respective examination and
for the computing device to also show the first operator, together with the established possible implementations of the examination task, the location of the settings of the medical imaging device and/or of the medical technology assessment of the respective examination and/or of the commercial assessment of the respective examination within the usual area in each case.

This means that it is possible for the first operator to recognize in a simple manner whether a specific possible implementation of the examination tasks lies within the usual framework. “Outliers” can readily be recognized in this way.

At least one embodiment is further achieved by a computer program. In accordance with at least one embodiment of the invention, the processing of the machine code by the computing device has the effect that the computing device carries out an inventive definition method.

At least one embodiment is further achieved a computing device. In accordance with the invention the computing device is programmed with at least one embodiment of an inventive computer program, so that, when in operation, it carries out at least one embodiment of an inventive definition method.

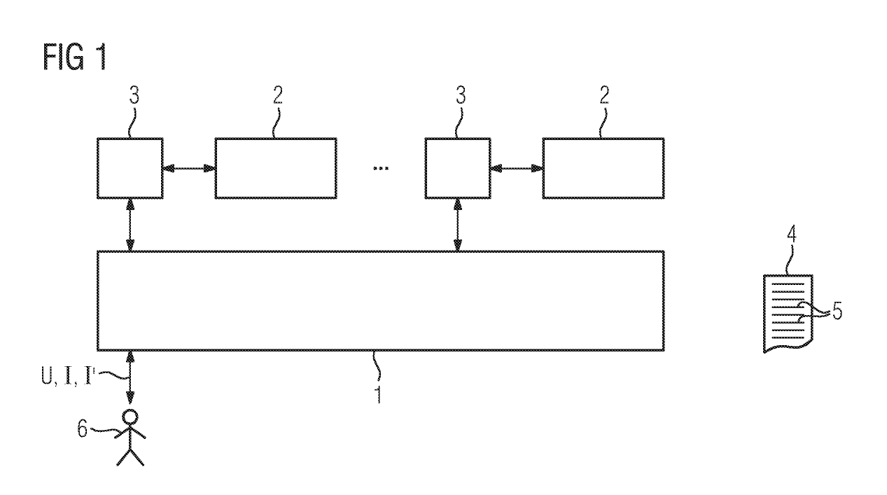

According to FIG. 1 a computing device 1 is arranged in a hospital environment. Within the hospital there is at least one medical imaging device 2, for example a CT scanner, an MR scanner, a C-arm x-ray system or a similar device. In technical circles such devices are usually referred to as a modality. A number of modalities 2 can also be present. It is even possible for a number of modalities of the same type to be present, for example a number of MR scanners. The modality 2 is controlled by a control device 3.

The computing device 1 is programmed with a computer program 4. The computer program 4 comprises machine code 5, which is able to be processed by the computing device 1. The processing of the machine code 5 by the computing device 1 has the effect that the computing device 1, when in operation, carries out a definition method for an implementation to be realized I′ of an examination task U, wherein the implementation to be realized I′ of the examination task U is to be brought about by the modality 2.

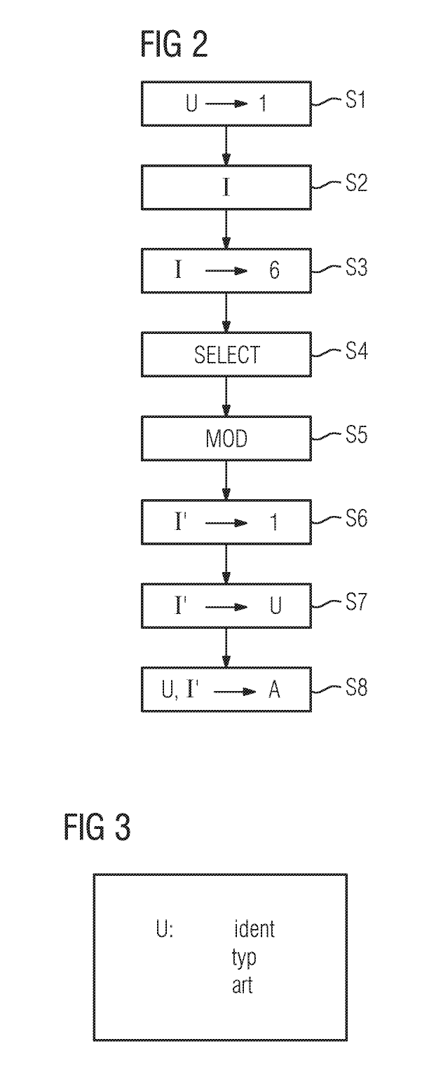

Within the framework of the definition method the computing device 1 first of all—see a step S1 in FIG. 2—accepts from a first operator 6—for example a doctor—an examination task U. According to FIG. 3 the examination task U comprises data ident, typ and art. The data ident is data that individualizes a person to be examined. It can for example be the name (if necessary broken down into first name and last name), an address, a date of birth, a number allocated only once and more besides. The data typ is data that typifies the person to be examined. The data typ can for example comprise the age, the gender, the weight, the size of the person to be examined and more besides. The data art defines the type of examination. The data art can for example define a region of the body of the person to be examined for example and a diagnosis aim, for example whether the person to be examined is to be examined for a broken bone, bleeding or a cancer. Often the preferred modality 2 for the examination will also be determined or at least marked as preferred by way of the data art, under some circumstances, with a number of possible modalities 2 of the same type, even a quite specific one of these modalities 2.

Certain crossovers can occur between the data ident and typ. Thus for example the gender can be used both for identification or individualization of the person to be examined and also for typifying this person. The same applies to the date of birth, which in conjunction with the current date, determines the age or the age class (=approximate age).

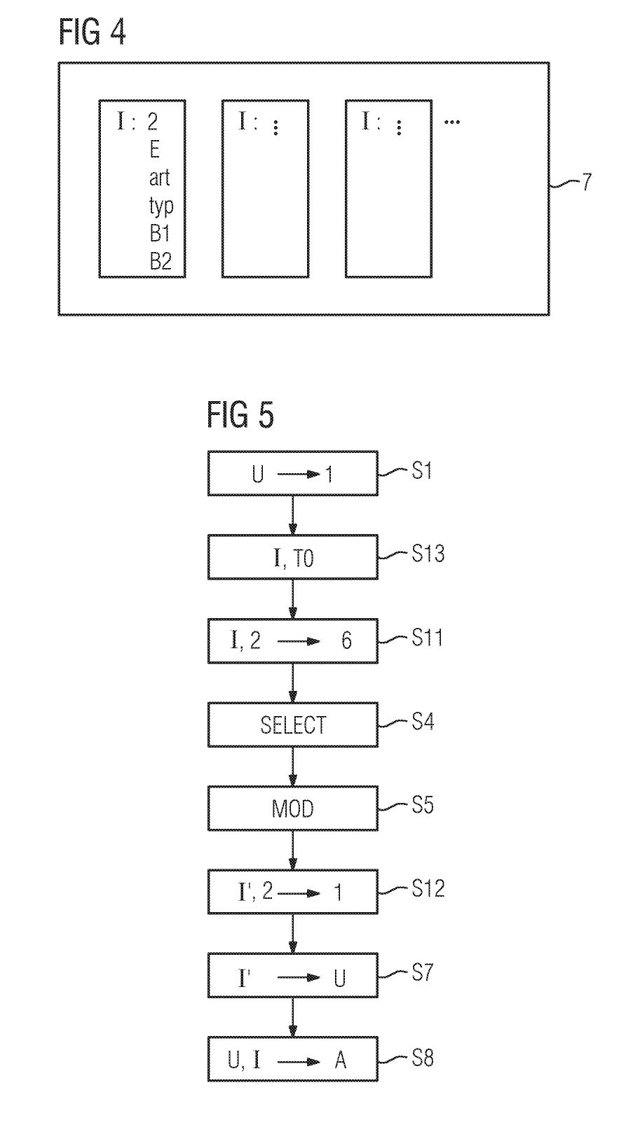

On the basis of the data typ and art the computing device 1 then, in a step S2, establishes—in the event of the specification of the modality 2, at least and preferably for this modality 2—possible implementations I of the examination task U. According to FIG. 4, the possible implementations I each comprise the modality 2 with which the examination task U is to be carried out, and the settings E of the corresponding modality 2. The settings E can comprise static and dynamic data, for example sequence protocols, voltages and currents to be used and more besides. The computing device 1 establishes the possible implementations I according to FIG. 1, while accessing a database 7. The database 7 contains, according to FIG. 4, for each implementation I stored in the database 7, the modality 2 and the settings E of the modality 2 as well as the type of examination and the data typ typifying the person to be examined. It further contains, for each implementation I, a medical technology assessment B1 and a commercial assessment B2. The medical technology assessment B1 gives information about the quality and the results that will be achieved when the modality 2 is operated with the settings E to examine a person with the characteristics characterized by the data typ for the type of examination specified for the implementation I. The commercial assessment B2 gives information about the cost-effectiveness of the operation of the modality 2. The commercial assessment B2 can be established in particular on the basis of the length of time that is required for carrying out the implementation I and thus for the examination.

The database 7 contains the implementations I, broken down into the said parameters. Thus, for specific data typ, art, not only is the modality 2 specified, but also the settings E and the assessments B1, B2. Preferably the implementations I are also indexed in the database 7, so that they can be found quickly and easily. The indexing is provided here at least for the data typ, art. In addition it can also be provided for a few or for all of the other parameters (modality 2, settings E and assessments B1, B2). Indexing is an established term for persons skilled in the art in the field of databases.

In a step S3 the computing device 1 specifies the established possible implementations I of the examination task U to the first operator 6 for selection. The specification is made together with the respective medical technology assessment B1 and the commercial assessment B2.

It is possible for the computing device 1 then to accept from the first operator 6, in a step S4, a pre-selection of one of the possible implementations I and, in a step S5, to accept modifications of the pre-selected implementation I. The steps S4 and S5 are only optional however. Regardless of whether the steps S4 and S5 are present, the computing device 1, in a step S6, accepts from the first operator 6 an implementation to be realized I′ of the examination task U however. The acceptance can in particular occur by (final) choice of one of the previously established (and if necessary modified) possible implementations I by the first operator 6.

In a step S7 the computing device 1 assigns the implementation to be realized I′ of the examination task U to the examination task U. Furthermore the computing device 1, in a step S8, updates a flowchart A for the modality 2 on the basis of the implementation I′. The flowchart A specifies the order in which the individual examination tasks U or the associated implementations I′ are to be carried out. The computing device 1 thus adds the newly included examination task U or the associated implementation I′ to the flowchart A. The updated flowchart A thus now also contains, in addition to implementations I′ of other examination tasks U already contained in flowchart A, the examination task U and the assigned implementation to be realized I′.

In many cases there is not just a single modality 2 present, but there are a number of modalities 2 present. In this case the method of operation of FIG. 2 is modified in a manner that will be explained in greater detail in conjunction with FIG. 5.

In accordance with FIG. 5, on the one hand step S3 is replaced by a step S11. The step S11 essentially corresponds to the step S3. In addition to the specification of the established possible implementations I, the computing device 1 also outputs, in step S11, information about the respective modality 2 for which the respective possible implementation I is valid. The possible implementations I will thus be specified to the first operator 6 broken down according to the respective modality 2. Furthermore in this case the step S6 is replaced by a step S12. In step S12 the computing device 1, in addition to the specifications of step S6, accepts from the first operator 6 a specification about the modality 2 with which the examination task U is to be carried out.

Preferably the computing device 1 further establishes, in accordance with the diagram shown in FIG. 5, together with the possible implementations I in a step S13 (=replacement for step S2 of FIG. 2) on the basis of the flowchart for the respective modality 2 (as the flowchart A is available to the computing device 1 before the acceptance of the examination task U) for the implementations I also in each case a possible beginning of implementation T0. The respective beginning of implementation T0 specifies the point in time at which the respective implementation I of the examination task U can be started at the respective modality 2. In this case the computing device 1 also shows to the first operator 6, together with the established possible implementations I of the examination task U, the respective possible beginning of implementation T0.

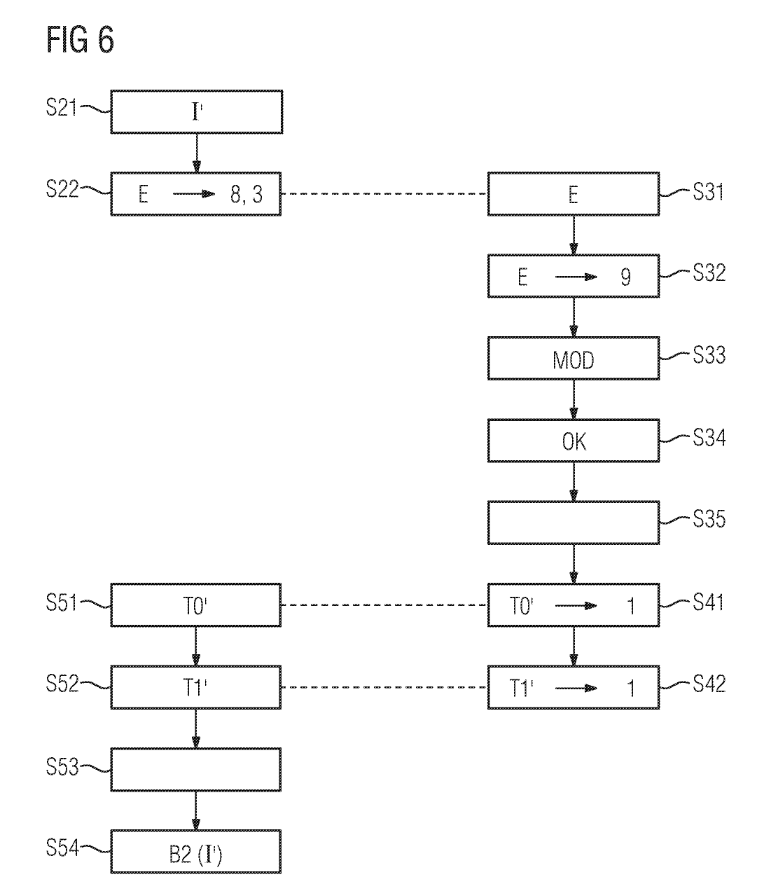

Via the computing device 1 not only is the flowchart A established (inter alia) for the respective modality 2. On the basis of the flowchart A for the (if necessary respective) modality 2, the computing device 1 is also in a position, as depicted in the diagram in FIG. 6, in a step S21, to establish for the respective modality 2 the next implementation to be realized I′ in each case. This means that the computing device 1 is also in a position, in a step S22, to specify to a second operator 8 the associated settings E of the respective modality 2 for the next implementation to be realized I′. The second operator 8 can for example be a medical technology assistant, who is operating and controlling the corresponding modality 2 via its control device 3.

It is possible for there to be a direct specification to the second operator 8 in step S22. Often however the computing device 1 has a data link to the control device 3 for the modality 2. In this case the computing device 1 can transfer the settings E to the control device 3. The control device 3 in this case accepts the settings E in a step S31 and outputs them in a step S32 via an operating terminal 9 to the second operator 8. The second operator 8 is responsible for the actual settings E of the modality 2. They are therefore in a position to modify the settings E in a step S33 and to confirm them in a step S34. Only as a result of the confirmation does the control device 3 accept the respective settings E as final settings E. As a general rule, although the second operator 8 is actually in a position, in step S33, to make modifications, realistically speaking however, they do not make any modifications. In a step S35 the control device 3 then controls the modality 2 according to the settings E.

According to the diagram shown in FIG. 6 further embodiments are possible. Thus it is possible in particular for the control device 3 to transfer the actual beginning of the execution of the examination task U—i.e. the point in time T0′ of the beginning of the implementation to be realized I′—to the computing device 1 in a step S41. In this case the computing device 1 accepts the actual beginning of implementation T0′ of the examination task U by the modality 2 in a step S51. In addition a step S42 can be present, in which the control device 3 transfers to the computing device 1 the actual end T1′ of the execution of the examination task U—i.e. the point in time of the end of the implementation to be realized I′. In this case the computing device 1 accepts the actual end of implementation T1′ by the modality 2 in a step S52. In a step S53 the computing device 1 can then estimate the period of time required by the modality 2 for carrying out the implementation I′. In a step S54 the computing device 1 can then update the commercial assessment B2 on the basis of the period of time.

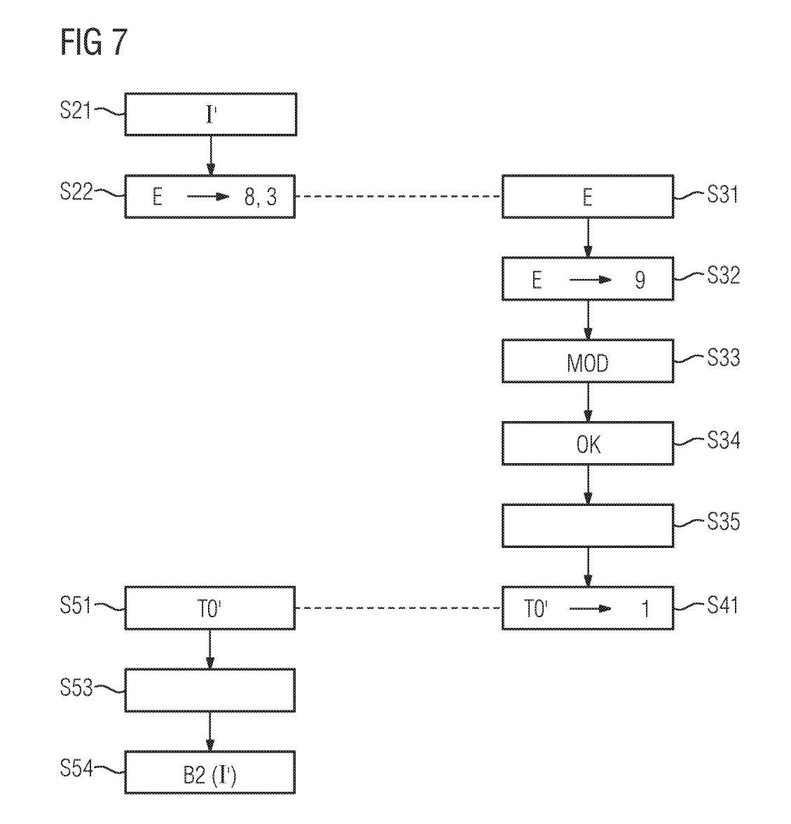

As an alternative. as shown in the diagram in FIG. 7, it is possible for the steps S42 and S52 to be omitted. In this case the computing device 1, in step S53, can likewise estimate a period of time required for carrying out of an implementation I′ by the modality 2. Unlike the method of operation in FIG. 6 however, this period of time is not related to the implementation I′ to which the beginning of implementation T0′ applies, but to the implementation I′ immediately preceding this implementation I′.

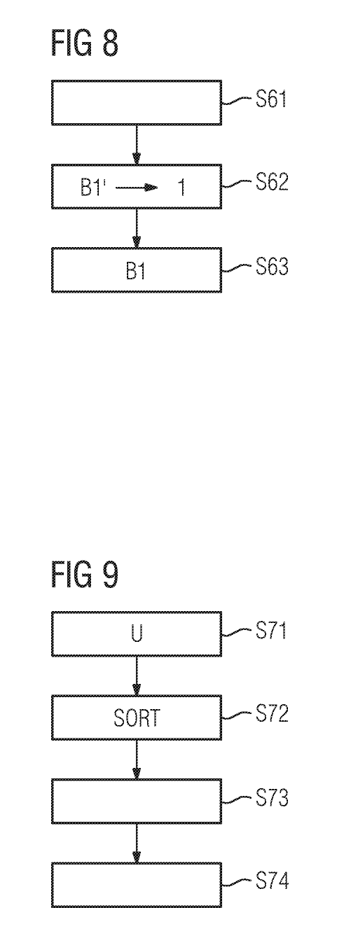

As well as the commercial assessment B2, the medical technology assessment B1 can also be updated. In this case the computing device 1 can for example, as depicted in the diagram in FIG. 8, in a step S61, evaluate the data acquired via the modality 2—in particular images. As an alternative or in addition the computing device 1, in a step S62, can accept from a third operator 10 (who can be identical to the first operator 6 for example) an assessment B1′. Then, in a step S63, the medical technology assessment B1 is updated on the basis of the evaluation of step S61 and/or assessment B1′. The method of operation of FIG. 8 can be combined for example as required with the method of operation of FIG. 6 or FIG. 7, in particular can follow on from the methods of operation explained for these figures.

To establish the commercial assessments B2 for the individual possible implementations I stored in the database 7 for the first time, as depicted in the diagram in FIG. 9 for example, the computing device 1, in a step S71, can access datasets of examination tasks U already carried out. The examination tasks U already carried out, as a general rule, comprise in each case the data typ typifying the person to be examined, the type of examination, the modality 2 and the settings E of the modality 2 as well as the start times T0′ of the respective examination task U. The examination tasks U already carried out, although they are present as a rule, are not chained to one another. The computing device 1 therefore compares, in particular in a step S72, the start times T0′ with one another and establishes in this way—i.e. on the basis of the start times T0′—the order of the examination tasks U already carried out. Then the computing device 1, in a step S73, establishes on the basis of the difference between immediately consecutive start times T0′, a respective estimated implementation duration for the respective examination task U already carried out. Finally the computing device 1, in a step S74, establishes the respective commercial assessment B2. It takes the implementation durations established in step S73 into consideration here.

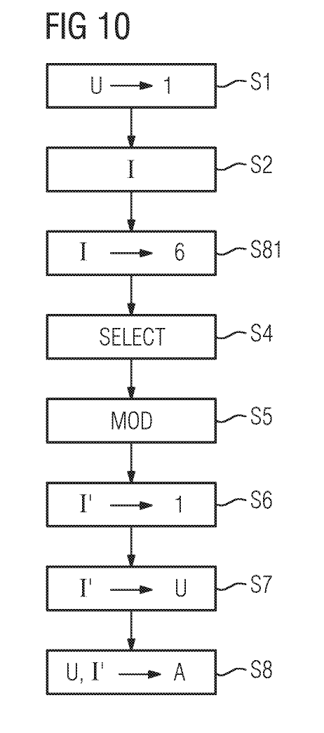

In many cases the database contains further data as well as typifying data typ, broken down according to the person to be examined, type of examination and modality 2. In particular it can contain a usual area in each case for the assigned data for the further implementations of a respective possible implementation—i.e. for the settings E of the modality 2, the medical technology assessment B1 of the respective examination and/or the commercial assessment B2 of the respective examination. In this case, as depicted in the diagram in FIG. 10, the step S3 of FIG. 2 is replaced by a step S81. The step S81 corresponds at its heart to step S3. However in step S81 the first operator 6 is shown not only the established possible implementations I of the examination task U, but in addition the location of the settings E of the modality 2 and/or of the medical technology assessment B1 and/or of the commercial assessment B2 within the usual area in each case.

In summary, at least one embodiment of the present invention thus relates to the following subject matter:

A computing device 1 accepts from a first operator 6 an examination task U to be carried out by a medical imaging device 2 (modality 2). The examination task U comprises data ident individualizing a person to be examined, data typ typifying the person and data art defining the type of examination. The computing device 1 accepts from the first operator 6 an implementation to be realized I′ of the examination task U and assigns this implementation I′ to the examination task U. The computing device 1 establishes on the basis of the data typ typifying the person and data art defining the type of examination, while accessing a database 7, possible implementations I of the examination task U. The database 7 contains typifying data typ broken down into the person to be examined, type of examination, modality 2 and settings E of the modality 2, a medical technology assessment B1 of the respective examination and a commercial assessment B2 of the respective examination in each case. The computing device 1 specifies the established possible implementations I to the operator 6, together with the two assessments B1, B2 for selection. On the basis of the implementation to be realized I′, accepted by the operator 6, it updates a flowchart A for the modality 2, so that the flowchart A, now contains, in addition to implementations I′ of other examination tasks U already contained in the flowchart A, what is now the examination task U and the assigned implementation to be realized I′.

Embodiments of the present invention has many advantages. In particular the operation of the modalities 2 can be optimized in a simple and reliable way both in respect of economic operation and also in respect of medical technology. Furthermore the comparison with the usual methods of operation in the hospital of each individual operation step can be undertaken both before and also after the examination task U has been carried out. The appraisal of the implementation to be realized I′ can be conducted long before it is carried out, in particular even outside the respective modality 2. The optimization of the settings E can be done in both dimensions (medical technology and commercial). The expected beginning of implementation T0 can be established beforehand and taken into consideration in the choice of the implementation to be realized I′. After the examination task U has been carried out there can be feedback for optimization.

Although the invention has been illustrated and described in greater detail by the preferred exemplary embodiments, the invention is not restricted solely to the disclosed examples and other variations can be derived herefrom by the person skilled in the art, without departing from the scope of protection of the invention.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for an implementation of an examination task by a medical imaging device, the method comprising:
accepting, via a computing device and from a first operator, the examination task to be carried out by the medical imaging device, the examination task including data individualizing a person to be examined, data typifying the person to be examined and data defining a type of examination method;
accepting, via a computing device and from a first operator, an implementation of the examination task to be realized and assigning the implementation, of the examination task to be realized, to the examination task;
breaking down the computing device, according to the data typifying the person to be examined, and data defining the type of examination of the examination task, while accessing a database, to establish possible implementations of the examination task, the database containing data typifying the person, broken down according to the person to be examined, type of examination, medical imaging device and settings of the medical imaging device, a medical technology assessment of the respective examination and a commercial assessment of the respective examination;
specifying, via the computing device, the possible implementations of the examination task established to the first operator together with a respective medical technology assessment and the commercial assessment for selection; and
updating via the computing device, based upon the implementation of the examination task to be realized accepted from the first operator, a flowchart for the medical imaging device, the flowchart, in addition to implementations of other examination tasks already contained in the flowchart, containing the examination task and the assigned implementation of the examination task to be realized.

2. The method of claim 1, further comprising:
establishing, via the computing device and based upon the flowchart, a next respective implementation to be realized by the medical imaging device; and
specifying settings of the medical imaging device, for the next respective implementation to be realized, to a second operator.

3. The method of claim 2, wherein the computing device includes a data link to a control device of the medical imaging device and wherein the computing device is configured to specify, to the control device, the settings of the medical imaging device for the next respective implementation to be realized by the medical imaging device, based upon a confirmation by the second operator.

4. The method of claim 1, wherein the computing device is configured to specify the established possible implementations of the examination task to the first operator, broken down according to the respective medical imaging device, and the computing device, within framework of the examination task, is further configured to accept a specification from the first operator, as to the medical imaging device with which the examination task is to be carried out.

5. The method of claim 1, wherein the computing device is configured to establish, based upon the flowchart for the medical imaging device and as available to the computing device before the acceptance of the examination task for the respective possible implementations of the examination task, a possible beginning of implementation, at which the respective possible implementation of the examination task is startable, and wherein the computing device is further configured to show the first operator, together with the established possible implementations of the examination task, the possible beginning of implementation.

6. The method of claim 1, wherein the computing device is configured to accept, to accept the implementation to be realized from the first operator, a choice of one of the possible implementations with or without modification of the respective possible implementation.

7. The method of claim 1, wherein the computing device is configured to accept an actual beginning of implementation of the examination task by the medical imaging device and to accept an actual end of implementation of the examination task by the medical imaging device or the actual beginning of implementation of the next examination task by the medical imaging device, wherein the computing device is further configured to estimate a period of time required for execution of the implementation of the examination task by the medical imaging device and wherein the computing device is further configured to update the commercial assessment based upon the period of time estimated.

8. The method of claim 1, wherein the computing device is configured to at least one of
evaluate the data acquired via the medical imaging device and
accept an assessment from a third operator and
wherein the computing device is configured to update the medical technology assessment, based upon at least one of the data evaluated and the assessment, accepted from the third operator.

9. The method of claim 1, wherein the computing device is configured to
access datasets of examination tasks already carried out, the examination tasks already carried out each including data typifying the person examined, the type of examination, the medical imaging device, the settings of the medical imaging device and start times of the respective examination tasks,
establish, based upon the start times, an order of the examination tasks already carried out and establish, for the respective examination task already carried out, an estimated implementation duration; and
take into consideration, the estimated implementation durations in establishing the commercial assessment.

10. The method of claim 1, wherein the database contains a usual area, each respectively broken down according to data typifying the person to be examined, type of examination and medical imaging device for settings of at least one of the medical imaging device, the medical technology assessment of the respective examination and the commercial assessment of the respective examination; and
wherein the the computing device is configured to show the first operator, together with the established possible implementations of the examination task, a location of the settings of at least one of the medical imaging device, the medical technology assessment of the respective examination and the commercial assessment of the respective examination within a respective usual area.

11. A non-transitory computer readable medium, storing machine code executable by a computing device, the machine code being configured to cause, when executed by the computing device, the computing device to carry out the method of claim 1.

12. A computing device, comprising:
at least one processor configured to
accept, via the at least one processor and from a first operator, the examination task to be carried out by a medical imaging device, the examination task including data individualizing a person to be examined, data typifying the person to be examined and data defining a type of examination method;
accept, via the at least one processor and from a first operator, an implementation of the examination task to be realized and assigning the implementation, of the examination task to be realized, to the examination task;
break down the computing device, according to the data typifying the person to be examined, and data defining the type of examination of the examination task, while accessing a database, to establish possible implementations of the examination task, the database containing data typifying the person, broken down according to the person to be examined, type of examination, medical imaging device and settings of the medical imaging device, a medical technology assessment of the respective examination and a commercial assessment of a respective examination;
specify, via the at least one processor, the possible implementations of the examination task established to the first operator together with a respective medical technology assessment and the commercial assessment for selection; and
update, via the at least one processor, based upon the implementation of the examination task to be realized accepted from the first operator, a flowchart for the medical imaging device, the flowchart, in addition to implementations of other examination tasks already contained in the flowchart, containing the examination task and the assigned implementation of the examination task to be realized.

13. The method of claim 2, wherein the computing device is configured to specify the established possible implementations of the examination task to the first operator, broken down according to the respective medical imaging device, and the computing device, within framework of the examination task, is further configured to accept a specification from the first operator, as to the medical imaging device with which the examination task is to be carried out.

14. The method of claim 3, wherein the computing device is configured to specify the established possible implementations of the examination task to the first operator, broken down according to the respective medical imaging device, and the computing device, within framework of the examination task, is further configured to accept a specification from the first operator, as to the medical imaging device with which the examination task is to be carried out.

15. The method of claim 2, wherein the computing device is configured to establish, based upon the flowchart for the medical imaging device and as available to the computing device before the acceptance of the examination task for the respective possible implementations of the examination task, a possible beginning of implementation, at which the respective possible implementation of the examination task is startable, and wherein the computing device is further configured to show the first operator, together with the established possible implementations of the examination task, the possible beginning of implementation.

16. The method of claim 2, wherein the computing device is configured to accept, to accept the implementation to be realized from the first operator, a choice of one of the possible implementations with or without modification of the respective possible implementation.
